(19) United States
(12) Patent Application Publication
(10) Pub. No.: US 2025/0375167 A1
(43) Pub. Date: Dec. 11, 2025

(54) METHOD AND SYSTEM FOR DETERMINING POST-OPERATIVE IMAGES OF AN ANOMALY USING DEEP-LEARNING MODELS

(71) Applicant: L&T TECHNOLOGY SERVICES LIMITED, Chennai (IN)

(72) Inventors: RAKSHITH MANJUNATH, Bangalore (IN); NISARGA KRISHNEGOWDA, Mysore (IN)

(21) Appl. No.: 18/944,084

(22) Filed: Nov. 12, 2024

(30) Foreign Application Priority Data
Jun. 6, 2024 (IN) 202441044134

(51) Int. Cl. A61B 5/00; A61B 5/055; G16H 30/40

(52) U.S. Cl. CPC A61B 5/7267; A61B 5/055; G16H 30/40; A61B 2505/05; A61B 2576/00

(57) ABSTRACT

A method and a system for determining final post-operative images of an anomaly is disclosed. A processor inputs a pre-operative image of the anomaly to a first GAN, a second GAN, and a third GAN. Each of the first, second and third GANs are trained based on a training data that includes a training set of post-operative images of the anomaly corresponding to a training set pre-operative images of the anomaly. Further, a first post-operative image of the anomaly is determined from the first GAN, a second post-operative image of the anomaly is determined from the second GAN and a third post-operative image of the anomaly is determined from the third GAN. Two of the first, the second and the third post-operative images are selected based on a SSIM score of each of the first, the second and the third post-operative images.

### TECHNICAL FIELD

This disclosure relates generally to deep learning-based image generation model, and more particularly to a method and system for determining post-operative images of an anomaly using deep-learning models.

### BACKGROUND

Early detection of anomalies, such as tumours, ulcers, etc. in the human body is crucial for accurate diagnosis and effective treatment strategies. Analysing anomaly patterns in a patient's medical data provides valuable insights into the anomaly, assisting doctors in planning appropriate treatments based on the diagnosis. Conventional diagnostic methods such as laboratory tests, biopsies, and endoscopic procedures, etc. provide a current status of an anomaly. Additionally, predicting and analysing treatment outcomes based on the current status is based on practitioners' experience. Further, patients generally enquire practitioners about the prognosis to make informed decision about selection of a treatment plan. Patients are more likely to enquire about a prognosis of a treatment plan before opting for it specially in case of surgical treatment. Therefore, it becomes crucial for the doctors to provide an about accurate prognosis or outcome to the patient in order to convince them about the outcomes. There is no solution in industry to provide a tangible post-operative status of an anomaly other than practitioners' opinion based on their experience.

Therefore, there is a requirement for an efficient and effective methodology for determining post-operative status of an anomaly in order to provide an accurate prognosis of a suggested treatment.

### SUMMARY OF THE INVENTION

In an embodiment, a method of determining post-operative images of an anomaly is disclosed. The method may include simultaneously inputting, by a processor, pre-operative image of the anomaly, detected in an internal body part of a patient, to a first generative adversarial network (GAN), a second GAN and a third GAN. Further the method may include, determining, by the processor, a first post-operative image of the anomaly from the first generative adversarial network (GAN), a second post-operative image of the anomaly from the second GAN and a third post-operative image of the anomaly from the third GAN. In an embodiment, each of the first GAN, the second GAN and the third GAN are trained based on a training data comprising a training set of post-operative images of the anomaly corresponding to a training set of pre-operative images of the anomaly. The method may further include selecting, by the processor, two of the first post-operative image, the second post-operative image and/or the third post-operative image based on a Structural Similarity Index Measure (SSIM) score of each of the first post-operative image, the second post-operative image and the third post-operative image with respect to each other. Further the method may include, determining, by the processor, a final post-operative image of the anomaly by performing a pixel-wise aggregation of the selected two of the first post-operative image, the second post-operative image and/or the third post-operative image.

In another embodiment, a system of determining post-operative images of an anomaly is disclosed. The system may include a processor, a memory communicably coupled to the processor, wherein the memory may store processor-executable instructions, which when executed by the processor may cause the processor to simultaneously input pre-operative image of the anomaly, detected in an internal body part of a patient, to a first generative adversarial network (GAN), a second GAN and a third GAN. Further, the processor may determine a first post-operative image of the anomaly from the first generative adversarial network (GAN), a second post-operative image of the anomaly from the second GAN and a third post-operative image of the anomaly from the third GAN. In an embodiment, each of the first GAN, the second GAN and the third GAN are trained based on a training data comprising a training set of post-operative images of the anomaly corresponding to a training set of pre-operative images of the anomaly. The processor may further select two of the first post-operative image, the second post-operative image and/or the third post-operative image based on a Structural Similarity Index Measure (SSIM) score of each of the first post-operative image, the second post-operative image and the third post-operative image with respect to each other. The processor may further determine a final post-operative image of the anomaly by performing a pixel-wise aggregation of the selected two of the first post-operative image, the second post-operative image and/or the third post-operative image.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of a post-operative image generation system, in accordance with some embodiments of the present disclosure.

FIG. 2 is a functional block diagram of a computing device of the post-operative image generation system of FIG. 1, in accordance with some embodiments of the present disclosure.

FIG. 3A illustrates an exemplary pre-operative MRI image of a brain including a tumour as an anomaly, in accordance with an exemplary embodiment of the present disclosure.

FIG. 3B illustrates a pre-operative image of the anomaly, in accordance with the exemplary embodiment of FIG. 3A.

FIG. 3C illustrates an exemplary final post-operative image of the anomaly, in accordance with the exemplary embodiments of FIG. 3A and FIG. 3B.

FIG. 4 illustrates a flow diagram of a methodology of determining post-operative image of an anomaly, in accordance with some embodiments of the present disclosure.

### DETAILED DESCRIPTION OF THE DRAWINGS

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered exemplary only, with the true scope being indicated by the following claims. Additional illustrative embodiments are listed.

Further, the phrases “in some embodiments”, “in accordance with some embodiments”, “in the embodiments shown”, “in other embodiments”, and the like mean a particular feature, structure, or characteristic following the phrase is included in at least one embodiment of the present disclosure and may be included in more than one embodiment. In addition, such phrases do not necessarily refer to the same embodiments or different embodiments. It is intended that the following detailed description be considered exemplary only, with the true scope and spirit being indicated by the following claims.

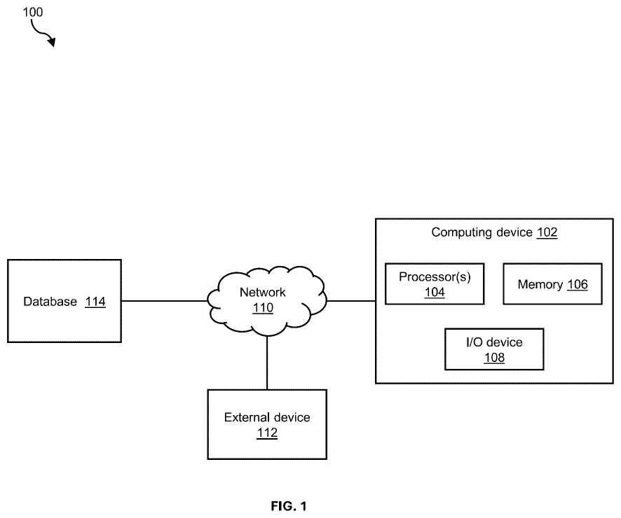

Referring now to FIG. 1, illustrates a block diagram of a post-operative image generation system 100 for generating a post-operative images of an anomaly detected in an internal body part of a patient, in accordance with some embodiments of the present disclosure.

The post-operative image generation system 100 may be implemented as a plurality of distributed cloud-based resources by use of several technologies that are well known to those skilled in the art. Other examples of implementation of the post-operative image generation system 100 may include, but are not limited to, a web/cloud server, an application server, a media server, and a Consumer Electronic (CE) device.

The post-operative image generation system 100 may include a computing device 102, an external device 112, and a database 114 communicably coupled to each other through a wired or a wireless communication network 110. The computing device 102 may include a processor(s) 104, a memory 106 and an input/output (I/O) device 108. In an embodiment, examples of processor(s) 104 may include, but are not limited to, an Intel® Itanium® or Itanium 2 processor(s), or AMD® Opteron® or Athlon MP® processor(s), Motorola® lines of processors, Nvidia®, FortiSOC™ system on a chip processors or other future processors. In an embodiment, the memory 106 may store instructions that, when executed by the processor 104, and cause the processor 104 to determine post-operative image of an anomaly, as discussed in more detail below. In an embodiment, the memory 106 may be a non-volatile memory or a volatile memory. Examples of non-volatile memory may include but are not limited to, a flash memory, a Read Only Memory (ROM), a Programmable ROM (PROM), Erasable PROM (EPROM), and Electrically EPROM (EEPROM) memory. Further, examples of volatile memory may include but are not limited to, Dynamic Random Access Memory (DRAM), and Static Random-Access memory (SRAM).

In an embodiment, the I/O device 108 may comprise of variety of interface(s), for example, interfaces for data input and output devices, and the like. The I/O device 108 may facilitate inputting of instructions by a user communicating with the computing device 102. In an embodiment, the I/O device 108 may be wirelessly connected to the computing device 102 through wireless network interfaces such as Bluetooth®, infrared, or any other wireless radio communication known in the art. In an embodiment, the I/O device 108 may be connected to a communication pathway for one or more components of the computing device 102 to facilitate the transmission of inputted instructions and output results of data generated by various components such as, but not limited to, processor(s) 104 and memory 106.

In an embodiment, the database 114 may be enabled in a cloud or a physical database and may store pre-operative images and training data. In an embodiment, the training data may include a set of post-operative images of the anomaly corresponding to a training set of pre-operative images of the anomaly. In an embodiment, the database 114 may store data input by an external device 112 or output generated by the computing device 102. In an embodiment, the I/O device 108 may include an imaging device (not shown). In an embodiment, the imaging device may generally include one or more cameras, such as high-resolution camera or scanner such as, but not limited to, Magnetic Resonance Imaging (MRI), Positron Emission Tomography (PET), Transcranial Magnetic Stimulation (TMS), etc. to generate an input pre-operative image of an internal body part such as a brain, etc. Further, in some embodiment, the database 114 may include the images generated by the imaging device.

In an embodiment, the communication network 110 may be a wired or a wireless network or a combination thereof. The network 110 can be implemented as one of the different types of networks, such as but not limited to, ethernet IP network, intranet, local area network (LAN), wide area network (WAN), or a Metropolitan Area Network (MAN). Various devices in the post-operative image generation system 100 may be configured to connect to the communication network 110, in accordance with various wired and wireless communication protocols. Examples of such wired and wireless communication protocols may include, but are not limited to, a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols. Further network 110 can include a variety of network devices, including routers, bridges, servers, computing devices, storage devices, and the like.

In an embodiment, the computing device 102 may receive a request for determining post-operative images of an anomaly from an external device 112 through the network 110. In an embodiment, the computing device 102 and the external device 112 may be a computing system, including but not limited to, a smart phone, a laptop computer, a desktop computer, a notebook, a workstation, a portable computer, a personal digital assistant, a handheld, a scanner, or a mobile device. In an embodiment, the computing device 102 may be, but not limited to, in-built into the external device 112 or may be a standalone computing device.

In an embodiment, the computing device 102 may perform various processing for determining post-operative image of an anomaly. By way of an example, the computing device 102 may receive the pre-operative image of an anomaly detected in an internal body part of a patient and simultaneously input the pre-operative image to a first generative adversarial network (GAN), a second GAN and a third GAN simultaneously. In an embodiment, the computing device 102 may perform pre-processing of the pre-operative image. The pre-processing may include resizing the pre-operative image based on a predefined size to make the input image smaller and suitable for the computing device 102 to process further to generate post-operative image. Further, the pre-processing may include cropping the pre-operative image from the beginning and from the end in order to eliminate the extra slices of the image. The pixel intensity of the pre-operative image may be normalized between 0 and 1.

Further, the computing device 102 may use the first GAN, the second GAN and the third GAN to generate a first post-operative image of the anomaly from the first GAN, a second post-operative image of the anomaly from the second GAN, and a third post-operative image of the anomaly from the third GAN.

In an embodiment, each of the first GAN, the second GAN and the third GAN may be trained based on a training data that may include a training set of post-operative images of the anomaly corresponding to a training set of pre-operative images of the anomaly. The training set of pre-operative images of the anomaly may be determined from a set of pre-operative MRI images of the body part and the training set of post-operative images of the anomaly may be determined from a set of post-operative MRI images of the body part. In an embodiment, each of the set of pre-operative MRI image and each of the set of post operative MRI images capture the body part from a predefined angular view with respect to a central axis of the body part in order to capture all the required details from every angle.

Further, the training set of pre-operative image and the training set of post-operative images may be determined by using an encoder-decoder convolutional neural network. In an embodiment, the encoder-decoder convolutional neural network may be a pretrained network model that may detect the abnormality in the image of the body part captured in the set of pre-operative MRI images and the set of post-operative MRI images respectively. In an embodiment, the encoder-decoder convolution neural network may be, but not limited to, a UNet model that may involve encoder and decoder components. The encoder in the UNet model takes the the set of pre-operative MRI images and the set of post-operative MRI images as input and further progressively reduces resolution of the image while capturing important features and generates a low-resolution images. Further, the decoder takes the low-resolution images generated by the encoder and reconstructs a high-resolution version of the MRI images. Accordingly, this process produces the training set of pre-operative images and the training set of post-operative images having anomaly detected area segmented out from the set of pre-operative MRI images and the set of post-operative MRI images respectively.

Further, based on the training set of pre-operative image and the training set of post-operative image, the computing device 102 may train each of the first GAN, the second GAN, and the third GAN. The computing device 102 may then select two of the first post-operative image, the second post-operative image and/or the third post-operative image based on a

Structural Similarity Index Measure (SSIM) score between of each of the first post-operative image, the second post-operative image and the third post-operative image with respect to each other. A final post-operative image of the anomaly may be determined by the computing device 102 by performing a pixel-wise aggregation of the selected two of the first post-operative image, the second post-operative image and/or the third post-operative image.

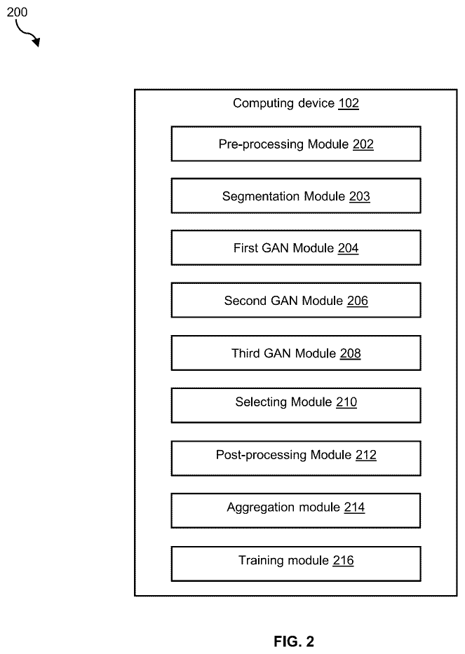

Referring now to FIG. 2, a functional block diagram of a computing device 102 of the post-operative image generation system 100 of FIG. 1, in accordance with some embodiments of the present disclosure. In an embodiment, the computing device 102 may include a pre-processing module 202, a segmentation module 203, a first GAN module 204, a second GAN module 206, a third GAN module 208, a selecting module 210, a post-processing module 212, an aggregation module 214 and a training module 216.

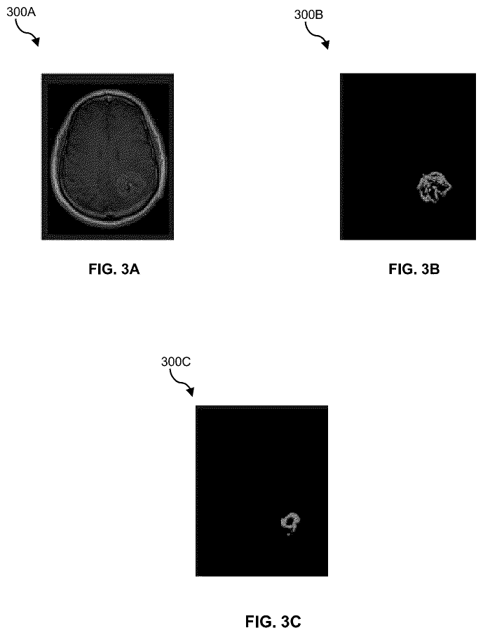

The computing device 102 may receive pre-operative image of the body part including an anomaly from the I/O device 108. Referring now to FIG. 3A, an exemplary pre-operative MRI image 300A of a brain including a tumour as an anomaly is illustrated, in accordance with an exemplary embodiment of the present disclosure. As will be appreciated, the anomaly may not be limited to a tumour and the body part in which the anomaly is determined may not be limited to brain of a patient and can be any part of the body.

Referring back to FIG. 2, the preprocessing module 202 may preprocess the pre-operative image of the brain including the anomaly 300A. The pre-processing may include resizing the image to a predefined size such as, but not limited to, 260×320 to adjust the input image as required for further processing. Further, pixel intensity values of the resized image may be normalized between 0 and 1 in order to adjust the local contrast in the image and bring out the clear regions or objects in the image. Further, the pre-processed image output by the preprocessing module 202 may be input to the segmentation module 203.

The segmentation module 203 may determine the pre-operative image of the anomaly from the pre-operative image of the body part including the anomaly 300A using an encoder-decoder convolutional neural network. The encoder-decoder convolutional neural network may be pretrained to detect an anomaly in a body part captured in the set of medical images. In an embodiment, the medical images may include MRI images as depicted in FIG. 3A. In an embodiment, the encoder-decoder convolution neural network may be a UNet model that may involve encoder and decoder components. The encoder in the UNet model takes the set of MRI images as input and may progressively reduce resolution of the image while capturing important features and generates a low-resolution image. Further, the decoder may take the low-resolution image generated by the encoder and reconstruct a high-resolution version of the MRI images. Accordingly, the encoder-decoder convolution neural network may produce an image having the anomaly segmented out from the input image. FIG. 3B illustrates a pre-operative image of the anomaly, in accordance with the exemplary embodiment of FIG. 3A. As shown in FIG. 3B, a pre-operative image of the tumor 300B segmented from the pre-operative image of the brain 300A is determined by the segmentation module 203.

The pre-operative image of the tumour 300B may then be input to each of the first GAN module 204, the second GAN module 206 and the third GAN module 208. In an embodiment, the pre-operative image of the tumour 300B may be fed into the first, the second and the third GAN modules 204-208 one by one or simultaneously.

Further, the first GAN module 204 may include the first GAN model and may determine a first post-operative image of the anomaly based on the pre-operative image 300B. In an embodiment the first GAN model, may be selected as, but not limited to, deep convolution generative adversarial network (DCGAN), External Classifier GAN, etc. The first GAN model of the first GAN module 204 may include a first generator model and first discriminator model. Further, the first generator model and the first discriminator model involve convolutional strides instead of pooling layers, BatchNorm for feature scale regulation, and LeakyReLU activation to prevent dead neurons. The pre-operative image of the tumour 300B may be input to the first generator model that may generate a first post-operative image of the anomaly. The first post-operative image of the anomaly may be input to the first discriminator model that may output a first probability score in a range of 0 to 1. In an embodiment, the first probability score may be indicative of a similarity of the first synthetic post-operative image with the training set of training set of post-operative images of the anomaly or the original post-operative image.

Further, the second GAN module 206 may use a second GAN model to determine a second post-operative image of the anomaly based on the pre-processed pre-operative image 300B. In an embodiment, the second GAN may be selected as, but not limited to, DCGAN, External Classifier GAN, etc. The second GAN may include a second generator model and second discriminator model. The second generator model may use a transposed convolution layer in contrast to the first generator model. Further, the second discriminator model may use convolutional strides, BatchNorm for feature scale regulation, and LeakyReLU activation similar to first GAN model of the first GAN module 204. It is to be noted that the use of transposed convolutional layers in the second generator model may allow for both up-sampling and details interpretation during the up-sampling process. The pre-operative image of the tumour 300B may be input to the second generator model that may generate a second post-operative image of the anomaly. Further, the second post-operative image of the anomaly may be input to the second discriminator model that may determine a second probability score in a range of 0 to 1 indicative of similarity of the second post-operative image of the anomaly with the training set of post-operative images of the anomaly or the original post-operative image.

The third GAN module 208 may determine a third post-operative image of the anomaly based on the pre-processed pre-operative image 300B, using a third GAN model. In an embodiment, the third GAN model may be selected as, but not limited to, Wasserstein generative adversarial network (WGAN). In an embodiment, the WGAN may offers training stability and a Wasserstein loss function that correlates with the quality of the generated images. The WGAN may include a third generator model and a critic model instead of a discriminator model to score images generated by the third generator model. The critic model may rely on Wasserstein-1 metric to minimize the distance between the data distributions. The pre-operative image of the tumour 300B may be input to the third generator model of the third GAN module 208 that may generate a third post-operative image of the anomaly. Further, the third post-operative image of the anomaly may be input to the critic model that may determine a continuous score indicating similarity of the third post-operative image of the anomaly with the training set of post-operative images of the anomaly or indicate the realness of the third post-operative image.

The selecting module 210 may select two post-operative images of the anomaly from the first post-operative image, the second post-operative image and the third post-operative image of the anomaly based on a structural similarity index measure (SSIM) score of each of the first post-operative image, the second post-operative image and the third post-operative image of the anomaly. In an embodiment, the SSIM score may be determined based on texture, luminance, and contrast of each of the first post-operative image, the second post-operative image and the third post-operative image. Further, the selecting module 210 may compare the SSIM score of each of the first post-operative image, the second post-operative image and the third post-operative image of the anomaly with respect to each other and select two images that have the highest SSIM score indicative of similarity to the original image.

The post-processing module 212 may determine edges of each of the two selected post-operative images of the anomaly by applying Sobel filters. On application of Sobel filters on the two selected post-operative images of the anomaly for capturing crucial information like horizontal and vertical edges and corresponding edge-mapped images are generated. Further, gaussian filter is applied to each of the corresponding edge-mapped images to determine corresponding two smoothened images in which the edges are more highlighted. It is to be noted that the pixel that are part of strong edges after applying gaussian filter are assigned higher weights. In an embodiment, weights assigned each pixel are determined based on equation (1) below:

$$W = gaussian\_matrix [ img\_ 1 ] + gaussian\_matrix [ img\_ 2 ] \qquad ( 1 )$$

It is to be noted that “img_1” and “img_2” are the two selected post-operative images.

Further, the aggregation module 212 may determine a final post-operative image of the anomaly by performing a pixel-wise aggregation of the selected two post-operative images each multiplied by weight “W” determined as per equation (1). Referring to FIG. 3C, an exemplary final post-operative image 300C of the detected anomaly, in accordance with the exemplary embodiments of FIG. 3A and FIG. 3B.

The training module 214 may train each of the first GAN model, the second GAN model and the third GAN model based on a training data. In an embodiment, the training data may include a training set of post-operative images of the anomaly corresponding to a training set pre-operative images of the anomaly. It is to be noted that the training set of pre-operative images of the anomaly may be determined from the set of pre-operative MRI images of the body part. Further, the training set of the post-operative images of the anomaly may be determined from the set of post-operative MRI images of the body part. In an embodiment, the set of pre-operative MRI images and the set of post-operative MRI images may be in a .dcm format. Further, each of the set of pre-operative MRI images and the set of post-operative MRI images may have 25 slices of height and width of 260×320.

In an embodiment, each of the set of pre-operative MRI images and each of the post-operative MRI images capture the body part, such as brain from a predefined angular view, such as anterior, posterior, top, etc. with respect to a central axis of the body part. Further, the pre-processing module 216 may preprocess each of the set of pre-operative MRI images 300A and each of the post-operative MRI images 300B. The pre-processing may include resizing the image to a predefined size such as, but not limited to, about 240×240. Further, pixel intensity values of each of the set of pre-operative MRI images and each of the post-operative MRI images may be normalized between 0 and 1 in order to adjust the local contrast in the image and bring out the clear regions or objects in the image.

Further, the pre-processed set of pre-operative MRI images and the pre-processed post-operative MRI images may be input to the segmentation module 218. The segmentation module 218 may determine the training set of the pre-operative images and the training set of post-operative images using an encoder-decoder convolutional neural network pretrained to detect the abnormality in the body part captured in the set of pre-operative MRI images and the post-operative MRI images respectively. It is to be noted that the training set of the pre-operative images and the training set of post-operative images may be masked images including the abnormality detected in the set of pre-operative MRI images and the pre-processed post-operative MRI images respectively. In an embodiment, the encoder-decoder convolution neural network may be, but not limited to, a UNet model.

The training set of post-operative images of the anomaly corresponding to the training set pre-operative images of the anomaly may be input to the GAN model to learn the differences between pre-operative images and post-operative images and to be able to generate the post-operative image of the anomaly based on the pre-operative image of the anomaly.

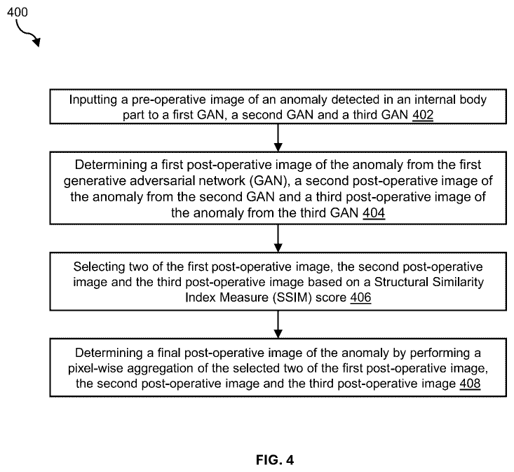

Referring to FIG. 4, a flow diagram 400 of a methodology of determining post-operative image of an anomaly, in accordance with some embodiments of the present disclosure. In an embodiment, the method 400 may include a plurality of steps that may be performed by the processor 104 to determine the post-operative image of the anomaly.

At step 402, a pre-operative image 300A of the anomaly such as a tumour, detected in the internal body such as brain, may be input simultaneously to a first GAN, a second GAN, and a third GAN. At step 404, a first post-operative image of the anomaly may be determined from the first GAN, a second post-operative image of the anomaly may be determined from the second GAN, and a third post-operative image of the anomaly may be determined from the third GAN.

In an embodiment, the first GAN, the second GAN and the third GAN may be trained based on a training data. The training data may include a training set of post-operative images of the anomaly corresponding to a training set pre-operative images of the anomaly.

Further at step 406, two of the first post-operative image, the second post-operative image and the third post-operative image may be selected based on the structural similarity index measure (SSIM) score of each of the first post-operative image, the second post-operative image, and the third post-operative image. In an embodiment, the SSIM score may be determined based on the texture, luminance and contrast of each of the first post-operative image, the second post-operative image and the third post-operative image.

Further, at step 408, a final post-operative image 300C of the anomaly may be determined by performing the pixel wise aggregation of the selected two of the first post-operative image, the second post-operative image, and the third post-operative image. In an embodiment, the pixel wise aggregation my include detection of the edges of the selected two of the first post-operative image, the second post-operative image, and the third post-operative image. Further, noise of the selected two of the first post-operative image, the second post-operative image, and the third post-operative image may be reduced using the gaussian filter in order to determine smoothened images. Further, weights may be assigned to pixels of the smoothened images. Further, the final post-operative image 300C may be determined based on the pixel wise aggregation of the smoothened images based on the weights assigned to pixels with higher intensity. In an embodiment, the higher weights may be assigned to pixels with higher intensity.

It is intended that the disclosure and examples be considered as exemplary only, with a true scope of disclosed embodiments being indicated by the following claims.

What is claimed is:

1. A method of determining post-operative image of an anomaly, comprising:
simultaneously inputting, by a processor, a pre-operative image of the anomaly, detected in an internal body part of a patient, to a first generative adversarial network (GAN), a second GAN and a third GAN;
determining, by the processor, a first post-operative image of the anomaly from the first generative adversarial network (GAN), a second post-operative image of the anomaly from the second GAN and a third post-operative image of the anomaly from the third GAN,
wherein each of the first GAN, the second GAN and the third GAN are trained based on a training data comprising a training set of post-operative images of the anomaly corresponding to a training set of pre-operative images of the anomaly;
selecting, by the processor, two of the first post-operative image, the second post-operative image and the third post-operative image based on a Structural Similarity Index Measure (SSIM) score of each of the first post-operative image, the second post-operative image and the third post-operative image; and
determining, by the processor, a final post-operative image of the anomaly by performing a pixel-wise aggregation of the selected two of the first post-operative image, the second post-operative image and the third post-operative image.

2. The method of claim 1, wherein the SSIM score is determined based on texture, luminance and contrast of each of the first post-operative image, the second post-operative image and the third post-operative image.

3. The method of claim 1, wherein the training set of pre-operative images of the anomaly are determined from a set of pre-operative MRI images of the body part,
wherein the training set of post-operative images of the anomaly are determined from a set of post-operative MRI images of the body part, and
wherein each of the set of pre-operative MRI images and each of the set of post-operative MRI images capture the body part from a predefined angular view with respect to a central axis of the body part.

4. The method of claim 3, wherein the training set of pre-operative images and the training set of post-operative images are determined using an encoder-decoder convolutional neural network pretrained to detect the abnormality in the body part captured in the set of pre-operative MRI images and the set of post-operative MRI images respectively.

5. The method of claim 3, wherein each of the set of pre-operative MRI images and the set of post-operative MRI images are resized to a predefined size.

6. The method of claim 3, wherein pixel intensity values of each of the set of pre-operative MRI images and the set of post-operative MRI images are normalized between 0 and 1.

7. The method of claim 1, wherein the third GAN measures a similarity score index of the third post-operative image with the training set of post-operative images of the anomaly by using a critic model.

8. The method of claim 1, wherein the determination of the final post-operative image comprises:
detecting, by the processor, edges of the selected two of the first post-operative image, the second post-operative image and the third post-operative image;
reducing, by the processor and upon detection of the edges, noise of the selected two of the first post-operative image, the second post-operative image and the third post-operative image using a gaussian filter to determine corresponding two smoothened images; and
assigning, by the processor, weights to pixels of the corresponding two smoothened images,
wherein the final post-operative image is determined based on pixel-wise aggregation of the corresponding two smoothened images based on the weights assigned to pixels with higher intensity.

9. A system for determining post-operative images of an anomaly, comprising:
a processor; and
a memory communicably coupled to the processor, wherein the memory stores processor-executable instruction, which, on executing by the processor cause the processor to:
simultaneously input pre-operative image of an anomaly detected in an internal body part to a first generative adversarial network (GAN), a second GAN and a third GAN;
determine a first post-operative image of the anomaly from the first GAN, a second post-operative image of the anomaly from the second GAN and a third post-operative image of the anomaly from the third GAN,
wherein each of the first GAN, the second GAN and the third GAN are trained based on a training data comprising a training set of post-operative images of the anomaly corresponding to a training set pre-operative images of the anomaly;
select two of the first post-operative image, the second post-operative image and the third post-operative image based on a Structural Similarity Index Measure (SSIM) score of each of the first post-operative image, the second post-operative image and the third post-operative image; and
determine a final post-operative image of the anomaly by performing a pixel-wise aggregation of the selected two of the first post-operative image, the second post-operative image and the third post-operative image.

10. The system of claim 9, wherein the SSIM score is determined based on texture, luminance and contrast of each of the first post-operative image, the second post-operative image and the third post-operative image.

11. The system of claim 9, wherein the training set of pre-operative images of the anomaly are determined from a set of pre-operative MRI images of the body part, wherein the training set of post-operative images of the anomaly are determined from a set of post-operative MRI images of the body part, and
wherein each of the set of pre-operative MRI images and each of the set of post-operative MRI images capture the body part from a predefined angular view with respect to a central axis of the body part.

12. The system of claim 11, wherein the training set of pre-operative images and the training set of post-operative images are determined using an encoder-decoder convolutional neural network pretrained to detect the abnormality in the body part captured in the set of pre-operative MRI images and the set of post-operative MRI images respectively.

13. The system of claim 11, wherein each of the set of pre-operative MRI images and the set of post-operative MRI images are resized to a predefined size.

14. The system of claim 11, wherein pixel intensity values of each of the set of pre-operative MRI images and the set of post-operative MRI images are normalized between 0 and 1.

15. The system of claim 9, wherein the third GAN measures a similarity score index of the third post-operative image with the training set of post-operative images of the anomaly by using a critic model.

16. The system of claim 9, wherein to determine the final post-operative image, the processor is configured to:
detect edges of the selected two of the first post-operative image, the second post-operative image and the third post-operative image;
upon the detection of the edges, reduce noise of the selected two of the first post-operative image, the second post-operative image and the third post-operative image using a gaussian filter to determine corresponding two smoothened images;
assign weights to pixels of the corresponding two smoothened images,
wherein the final post-operative image is determined based on pixel-wise aggregation of the corresponding two smoothened images based on the weights assigned to pixels with higher intensity.

17. A non-transitory computer-readable medium storing computer-executable instructions for determining post-operative image of an anomaly, the computer-executable instructions configured for:
simultaneously inputting a pre-operative image of the anomaly, detected in an internal body part of a patient, to a first generative adversarial network (GAN), a second GAN and a third GAN;
determining a first post-operative image of the anomaly from the first generative adversarial (GAN), a second post-operative image of the anomaly from the second GAN and a third post-operative image of the anomaly from the third GAN,
wherein each of the first GAN, the second GAN and the third GAN are trained based on a training data comprising a training set of post-operative images of the anomaly corresponding to a training set of pre-operative images of the anomaly;
selecting two of the first post-operative image, the second post-operative image and the third post-operative image based on a Structural Similarity Index Measure (SSIM) score of each of the first post-operative image, the second post-operative image and the third post-operative image; and
determining a final post-operative image of the anomaly by performing a pixel-wise aggregation of the selected two of the first post-operative image, the second post-operative image and the third post-operative image.

18. The non-transitory computer-readable medium of claim 17, wherein the SSIM score is determined based on texture, luminance and contrast of each of the first post-operative image, the second post-operative image and the third post-operative image.

19. The non-transitory computer-readable medium of claim 17, wherein the training set of pre-operative images of the anomaly are determined from a set of pre-operative MRI images of the body part,
wherein the training set of post-operative images of the anomaly are determined from a set of post-operative MRI images of the body part, and
wherein each of the set of pre-operative MRI images and each of the set of post-operative MRI images capture the body part from a predefined angular view with respect to a central axis of the body part.

20. The non-transitory computer-readable medium of claim 19, wherein the training set of pre-operative images and the training set of post-operative images are determined using an encoder-decoder convolutional neural network pretrained to detect the abnormality in the body part captured in the set of pre-operative MRI images and the set of post-operative MRI images respectively.
